(19) United States
(12) Patent Application Publication
(10) Pub. No.: US 2005/0136485 A1
(43) Pub. Date: Jun. 23, 2005

(54) Iterative one-pot oligosaccharide synthesis

(76) Inventors: Xuefei Huang, Sylvania, OH (US); Xin-Shan Ye, Beijing (CN)

Correspondence Address: MACMILLAN SOBANSKI & TODD, LLC;ONE MARITIME PLAZA FOURTH FLOOR, 720 WATER STREET, TOLEDO, OH, 43604-1619, US

(21) Appl. No.: 11/020,917

(22) Filed: Dec. 22, 2004

Related U.S. Application Data
(60) Provisional application No. 60/532,065, filed on Dec. 22, 2003.

(51) Int. Cl. G01N 33/53; C07H 5/04; C08B 37/00

(52) U.S. Cl. 435/7.1; 536/55.3; 536/124

(57) ABSTRACT

A process for synthesizing an oligosaccharide includes (a) activating a protected glycosyl donor with a promoter in the absence of a glycosyl acceptor to produce a reactive intermediate, the glycosyl donor having an activatable aglycon at the anomeric carbon; (b) adding a protected glycosyl donor/acceptor to the reactive intermediate to produce a new glycosyl donor, the glycosyl donor/acceptor having both an activatable aglycon at the anomeric carbon and a free hydroxyl group; (c) repeating steps (a) and (b) to add any additional protected glycosyl donor/acceptors; and (d) adding a protected glycosyl acceptor to produce the oligosaccharide, the glycosyl acceptor having a free hydroxyl group and a non-activatable aglycon at the anomeric carbon.

### CROSS-REFERENCE TO RELATED APPLICATION

This application claims the benefit of U.S. provisional application Ser. No. 60/532,065, filed on Dec. 22, 2003.

### BACKGROUND OF THE INVENTION

This invention relates in general to carbohydrate chemistry, and in particular to an improved process for oligosaccharide synthesis.

Carbohydrates have been recognized to play significant roles in numerous physiological events, such as immunological response, inflammation, cancer metastasis and bacterial/viral infection. However, the lack of general methods for oligosaccharide assembly has greatly hampered glycobiology research. Methodologies such as solution based one-pot synthesis and solid phase synthesis have been developed in the past decade for facilitating oligosaccharide synthesis. One-pot strategy refers to methods in which several glycosylation steps are carried out in one reaction vessel without purification of intermediates. Currently, this has been primarily accomplished by sequentially reacting glycosyl building blocks with decreasing anomeric reactivities (Scheme 1a). The reactivities of building blocks are controlled either through different protective groups on the glycons or different anomeric aglycon groups. The one-pot oligosaccharide synthesis is a great advance for glyco-assembly. By removing the need for tedious intermediate purification, the procedures for oligosaccharide assemblies are simplified. However, the requirement for extensive protective group manipulation to adjust the reactivity and multiple activators are serious drawbacks of this reactivity based one-pot approach.

The other growing trend for oligosaccharide synthesis is solid phase synthesis (Scheme 1b). Advantages of conducting oligosaccharide synthesis on solid phase are obvious: it eliminates the need for intermediate purification and possesses great potential for automation. However, the complexity of oligosaccharide assembly coupled with unpredictability of glycosylation reactions on polymer support and the difficulty of following progress of the reaction has significantly hindered the development of oligosaccharide synthesis on solid phase.

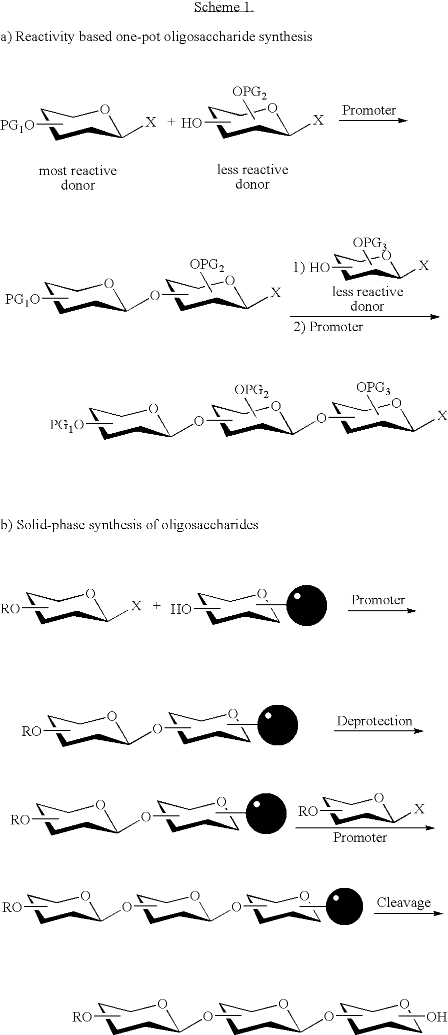

Therefore, it would be highly desirable to provide an improved process for one-pot oligosaccharide synthesis that combines the advantages of one-pot solution synthesis and solid phase synthesis and avoids their disadvantages.

### SUMMARY OF THE INVENTION

This invention relates to a process for synthesizing an oligosaccharide. The oligosaccharide contains three or more glycosyl units linked to one another by glycosidic linkages, starting with a first glycosyl unit at a nonreducing end, concluding with a final glycosyl unit at a reducing end, and including one or more intermediate glycosyl units sequentially arrayed between the first and final glycosyl units. The process comprises the steps of:
(a) synthesizing a protected glycosyl donor corresponding to the first glycosyl unit, one or more protected glycosyl donor/acceptors corresponding to each of the intermediate glycosyl units, and a protected glycosyl acceptor corresponding to the final glycosyl unit, the protected glycosyl donor having an activatable aglycon at the anomeric carbon, the protected glycosyl donor/acceptors each having both an activatable aglycon at the anomeric carbon and a free hydroxyl group, the glycosyl acceptor having a free hydroxyl group and a non-activatable aglycon at the anomeric carbon;
(b) activating the glycosyl donor with a promoter in the absence of a glycosyl acceptor to produce a reactive intermediate;
(c) adding one of the protected glycosyl donor/acceptors to the reactive intermediate to produce a new glycosyl donor;
(d) repeating steps (b) and (c) to add any additional protected glycosyl donor/acceptors; and (e) adding the protected glycosyl acceptor to produce the oligosaccharide.

In a related embodiment of the invention, the process for synthesizing the oligosaccharide comprises the steps of:
(a) activating a protected glycosyl donor with a promoter in the absence of a glycosyl acceptor to produce a reactive intermediate, the glycosyl donor corresponding to the first glycosyl unit and having an activatable aglycon at the anomeric carbon;
(b) adding a protected glycosyl donor/acceptor to the reactive intermediate to produce a new glycosyl donor, the glycosyl donor/acceptor corresponding to one of the one or more intermediate glycosyl units and having both an activatable aglycon at the anomeric carbon and a free hydroxyl group;
(c) repeating steps (a) and (b) to add any additional protected glycosyl donor/acceptors corresponding to the one or more intermediate glycosyl units; and
(d) adding a protected glycosyl acceptor to produce the oligosaccharide, the glycosyl acceptor corresponding to the final glycosyl unit and having a free hydroxyl group and a non-activatable aglycon at the anomeric carbon.

In another embodiment, the invention relates to a process for synthesizing an oligosaccharide and facilitating the purification of the desired oligosaccharide. The process comprises the steps of:
(a) synthesizing a protected glycosyl donor corresponding to the first glycosyl unit, one or more protected glycosyl donor/acceptors corresponding to each of the intermediate glycosyl units, and a protected glycosyl acceptor corresponding to the final glycosyl unit, the synthesizing of the glycosyl acceptor including a step of incorporating an affinity tag;
(b) activating the glycosyl donor with a promoter in the absence of a glycosyl acceptor to produce a reactive intermediate;
(c) adding one of the protected glycosyl donor/acceptors to the reactive intermediate to produce a new glycosyl donor;
(d) repeating steps (b) and (c) to add any additional protected glycosyl donor/acceptors; and
(e) adding the protected glycosyl acceptor to produce the oligosaccharide, the oligosaccharide including the affinity tag at the reducing end, the affinity tag facilitating purification of the oligosaccharide.

In another embodiment, the invention relates to a process for synthesizing an oligosaccharide library and incorporating the members of the library onto polymer. The process comprises the steps of:
(a) synthesizing a set of protected glycosyl donors, protected glycosyl donor/acceptors and protected glycosyl acceptors from different monosaccharides, the glycosyl donor/acceptors having at least one of 2-OH, 3-OH, 4-OH and 6-OH unprotected and glycosyl acceptors having an affinity tag and at least one of 2-OH, 3-OH, 4-OH and 6-OH unprotected;
(b) activating one of the glycosyl donors with a promoter in the absence of a glycosyl acceptor to produce a reactive intermediate;
(c) adding one of the protected glycosyl donor/acceptors to the reactive intermediate to produce a new glycosyl donor;
(d) repeating steps (b) and (c) to add any additional protected glycosyl donor/acceptors desired in the first oligosaccharide;
(e) adding one of the protected glycosyl acceptors to produce the first oligosaccharide containing the affinity tag at the reducing end;
(f) adding a polymer which the affinity tag containing oligosaccharide will be attached to; and
(g) repeating steps (b) through (f) using other combinations of the glycosyl donors, glycosyl donor/acceptors and glycosyl acceptors to produce other members of the oligosaccharide library.

In another embodiment, the invention relates to a process for synthesizing an oligosaccharide library. The process comprises the steps of:
(a) synthesizing a set of protected glycosyl donors, protected glycosyl donor/acceptors and protected glycosyl acceptors from different monosaccharides, the glycosyl donor/acceptors and glycosyl acceptors having at least one of 2-OH, 3-OH, 4-OH and 6-OH unprotected;
(b) activating one of the glycosyl donors with a promoter in the absence of a glycosyl acceptor to produce a reactive intermediate;
(c) adding one of the protected glycosyl donor/acceptors to the reactive intermediate to produce a new glycosyl donor;
(d) repeating steps (b) and (c) to add any additional protected glycosyl donor/acceptors desired in the first oligosaccharide;
(e) adding one of the protected glycosyl acceptors to produce the first oligosaccharide; and
(f) repeating steps (b) through (e) using other combinations of the glycosyl donors, glycosyl donor/acceptots and glycosyl acceptors to produce other members of the oligosaccharide library.

Various advantages of this invention will become apparent to those skilled in the art from the following detailed description of the preferred embodiments.

### DETAILED DESCRIPTION OF THE PREFERRED EMBODIMENTS

This invention provides a general iterative one-pot process for oligosaccharide synthesis which greatly expedites the preparation of oligosaccharides. The steps of the process are shown below in Scheme 2.

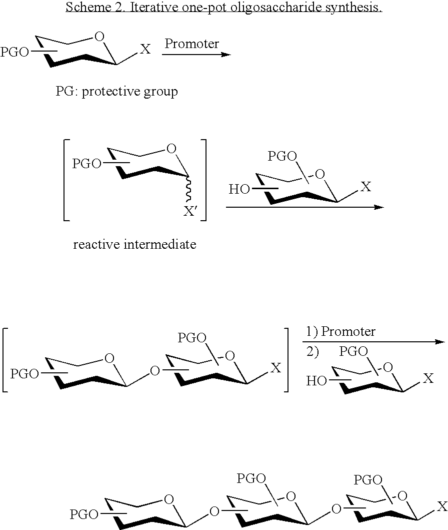

Overall, the process is useful for synthesizing an oligosaccharide which contains three or more glycosyl units linked to one another by glycosidic linkages, starting with a first glycosyl unit at a nonreducing end, concluding with a final glycosyl unit at a reducing end, and including one or more intermediate glycosyl units sequentially arrayed between the first and final glycosyl units. The oligosaccharide may also contain one or more glycosyl units branching from the main chain between the first and final glycosyl units.

The initial step of a preferred embodiment of the process is to synthesize a protected glycosyl donor corresponding to the first glycosyl unit, one or more protected glycosyl donor/acceptors corresponding to each of the intermediate glycosyl units, and a protected glycosyl acceptor corresponding to the final glycosyl unit. The protected glycosyl donor has an activatable aglycon at the anomeric carbon, the protected glycosyl donor/acceptors each have both an activatable aglycon at the anomeric carbon and a free hydroxyl group (at least one free hydroxyl group), and the glycosyl acceptor has a free hydroxyl group (at least one free hydroxyl group) and a non-activatable aglycon at the anomeric carbon. The protected glycosyl donor usually lacks a free hydroxyl group, but it may have a free hydroxyl group that is unreactive. As an alternative to synthesizing these glycosyl materials, they could be purchased already synthesized and used in the process steps described below.

Any suitable glycosyl donor can be used, including thioglycosides, glycosyl iodides, glycosyl fluoride or glycosyl sulfoxides synthesized from any of the common monosaccharides. In some embodiments of the invention, the glycosyl building blocks utilized are thioglycosides.

In the next step of the process, the glycosyl donor is activated with a promoter in the absence of a glycosyl acceptor. This produces a reactive intermediate having a reactive aglycon X′ at the anomeric carbon.

The promoter is stoichiometric in activating the glycosyl donor. Any suitable promoter can be used in the process. In some embodiments, thiophilic promoters, such as p-tolyl sulfenyl triflate (TolSOTf) as well as other aryl or alkyl sulfenyl triflates, can be utilized for thioglycoside building blocks.

The reactive intermediate generated by activation of the glycosyl donor with the promoter is sufficiently stable at the process temperature until the addition of the next building block of the oligosaccharide. However, the reactive intermediate is usually unstable at room temperature (21° C.). The reactive intermediate is reactive at the temperatures used in the process, which is typically below −30° C.

In the next step of the process, one of the protected glycosyl donor/acceptors is added to the reaction mixture. The glycosyl donor/acceptor reacts with the reactive intermediate to produce a new glycosyl donor (i.e., an oligosaccharide having an activatable aglycon X at the reducing end). Preferably, the original glycosyl donor and the glycosyl donor/acceptor contain identical activatable aglycons.

If the desired oligosaccharide is to contain more than one intermediate glycosyl unit, the prior two steps are repeated. Each iteration of the process steps will add one more intermediate glycosyl unit.

In the last step of the process, the protected glycosyl acceptor is added to the reaction mixture to produce a protected oligosaccharide. Thus, the process constructs a desired oligosaccharide in a one-pot manner, from the nonreducing end and progressing sequentially to the reducing end. Advantageously, the one-pot synthesis is carried out independent of anomeric reactivities of donors and donor/acceptors.

Preferably, all the reactions of the process are carried out in solution. The progress of the reactions in solution can be facilely monitored by routine methods.

The oligosaccharide product still contains the protective groups. Consequently, the process generally includes additional steps of removing all the protective groups to generate the desired oligosaccharide. The protective groups can be removed in any suitable manner.

Since protective groups can have significant effects on this glycosylation, glycosyl donors with different protective groups can be used to provide an optimum donor for each specific glycosidic linkage. Stereochemistry of the newly-formed glycosidic bond will be controlled by the presence/absence of a participating group on the C2 hydroxyl moiety. Advantageously, no protective group adjustments or aglycon modifications are necessary for intermediates in the process of the one-pot glycosylations.

The oligosaccharide can be purified by any suitable method, either before or after the removal of the protective groups. Advantageously, the purification of the final oligosaccharide is the only separation step necessary in the process. The process eliminates the need for tedious intermediate purification, thereby greatly simplifying the overall procedure and reducing time and effort required for glycoassembly.

In order to further expedite purification of the desired oligosaccharide product, in another embodiment of the invention, we use a “catch-and-release” protocol (Scheme 3) involving the incorporation of an affinity tag.

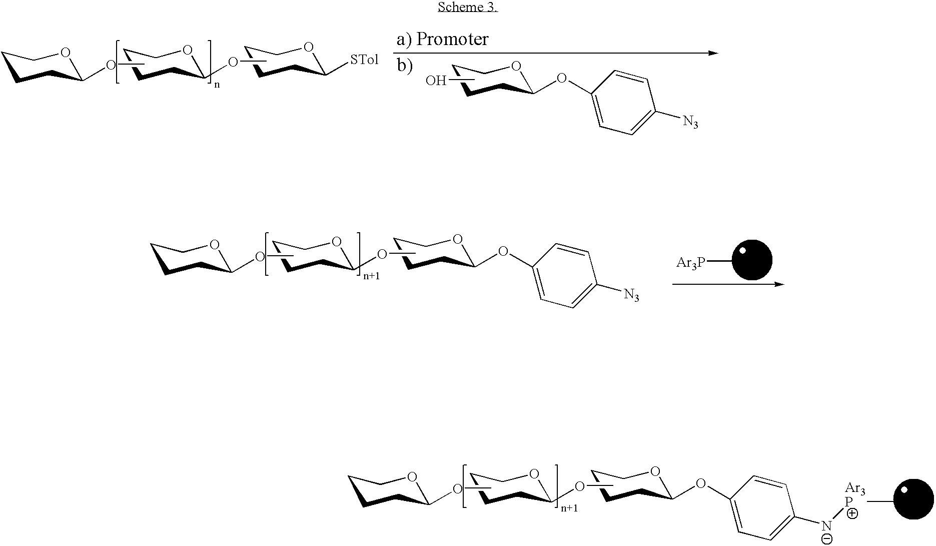

Generally, the affinity tag is incorporated by synthesizing the glycosyl acceptor with the affinity tag on its reducing end. Any suitable affinity tag can be used in the process to facilitate purification. The tag can be in various forms, such as a fluorous group, polyethylene glycol monomethyl ether or a reactive functional group which can be attached to a polymer. In some embodiments of the invention, the affinity tag is able to attach to an insoluble polymer. For example, an affinity tag such as an azide (Scheme 3) or a ketone/aldehyde can be used. An insoluble polymer can be added to the reaction solution after the oligosaccharide has been synthesized, so that the oligosaccharide attaches to the polymer. Any insoluble polymer suitable for attaching to the oligosaccharide can be used, for example a polymer containing phosphines. In other embodiments, the affinity tag is soluble, such as a fluorous chain or a polyethylene glycol chain. The polymer can be added in any suitable manner. For example, the polymer can be added in the form of polymer beads, and incubated in the reaction solution after the oligosaccharide has been synthesized.

In the embodiment shown in Scheme 3, upon completion of the last glycosidation reaction in which the azide tag is incorporated, polymer containing triarylphosphines is added to the reaction solution. Only compounds containing the azide moiety, which will mainly be the desired oligosaccharide product, will react with the polymer-bound triarylphosphines (by the Staudinger reaction). In the case of using ketone or aldehyde as affinity tags, polymers with hydrazide or aminooxy moieties can be utilized in order to facilitate isolation of the desired products.

The oligosaccharide attached to the polymer can be easily isolated from the reaction solution by filtration or another suitable method. The attached polymer makes isolation of the product much easier than isolating the oligosaccharide alone. After the product has been isolated, the oligosaccharide can be cleaved from the polymer by hydrolysis after washing away all the side products, and the polymer can be regenerated. Alternatively, the synthesized oligosaccharides can be deprotected while still attached to the polymer, which can be directly utilized for in vitro screening for carbohydrate-binding proteins and nucleic acids.

In another embodiment of the invention, oligosaccharide libraries are assembled and attached to polymer. Because the oligosaccharide synthesis process of the invention does not require tuning of glycosyl donor reactivities, only one set of building blocks is necessary for each type of glycosidic linkage. This greatly reduces the complexity of building block preparation, which allows facile construction of oligosaccharide libraries (Scheme 4).

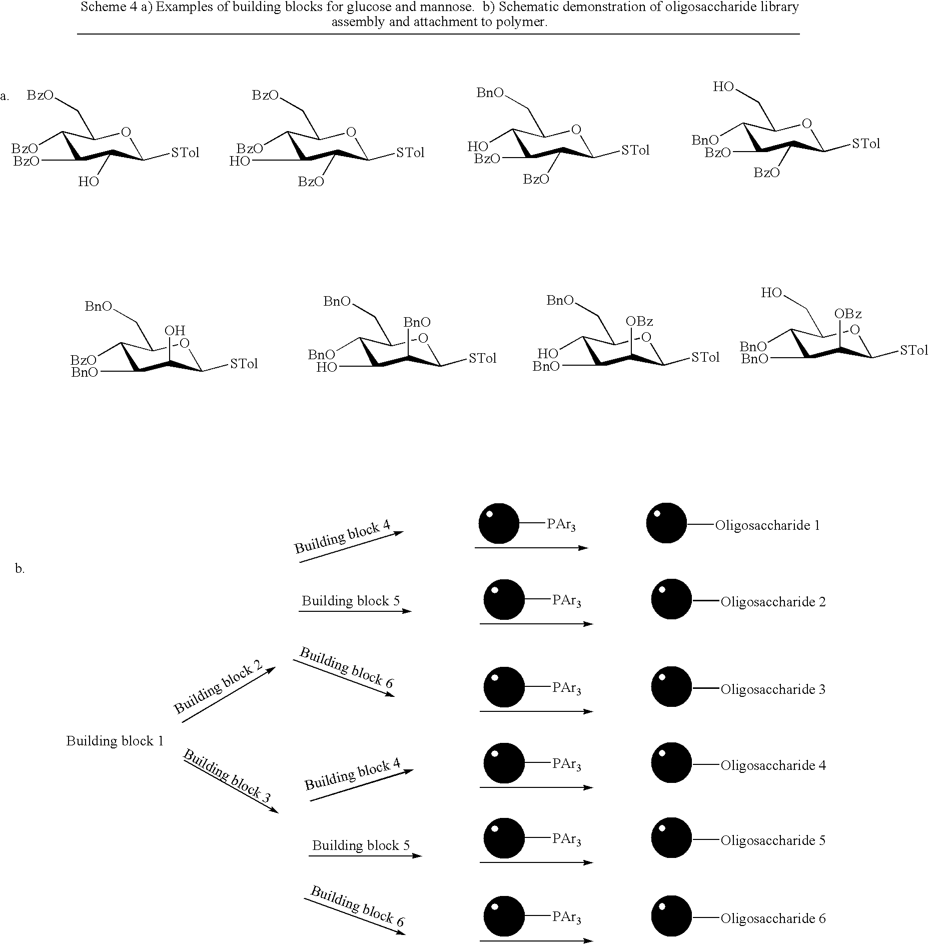

The process for synthesizing an oligosaccharide library includes a first step (a) of synthesizing a set of protected glycosyl donors, protected glycosyl donor/acceptors and protected glycosyl acceptors from different monosaccharides. For example, they can be synthesized from the common monosaccharides, e.g., glucose, mannose, galactose, N-Ac-glucosamine, and N-Ac-galactosamine. The glycosyl donor/acceptors have at least one of 2-OH, 3-OH, 4-OH and 6-OH unprotected and the glycosyl acceptors have an affinity tag and at least one of 2-OH, 3-OH, 4-OH and 6-OH unprotected.

Then the process is like that described above, except that the process steps are repeated with different building blocks to produce different oligosaccharides for the library. Specifically, the process includes the steps of: (b) activating one of the glycosyl donors with a promoter in the absence of a glycosyl acceptor to produce a reactive intermediate; (c) adding one of the protected glycosyl donor/acceptors to the reactive intermediate to produce a new glycosyl donor; (d) repeating steps (b) and (c) to add any additional protected glycosyl donor/acceptors desired in a first oligosaccharide; (e) adding one of the protected glycosyl acceptors to produce the first oligosaccharide; (f) adding a polymer and attaching the affinity tag containing oligosaccharide to the polymer and (g) repeating steps (b) through (f) using other combinations of the glycosyl donors, glycosyl donor/acceptors and glycosyl acceptors to produce other members of the oligosaccharide library. The process is useful for synthesizing a wide variety of oligosaccharides including linear and branched oligosaccharides.

Alternatively, oligosaccharide libraries can be assembled without attaching the affinity tag. The process is the same as described above except that step (f) is left out.

Combinatorial synthesis can be carried out with these building blocks, and oligosaccharide libraries assembled in this manner can be utilized in searches for carbohydrate ligands for proteins or nucleic acids.

In summary, the present invention provides a novel approach of iterative one-pot glycosylation for the efficient preparation of oligosaccharides. Oligosaccharides can be synthesized in good yields without any intermediate purification. Furthermore, this approach obviates the need for the extensive protective group adjustment required for traditional reactivity based one-pot synthesis, significantly reducing the amount of time needed for building block preparation. A catch-and-release protocol may be used to further expedite purification of the desired oligosaccharide. In addition, this approach only requires a single glycosylation method for all coupling steps, thus greatly streamlining oligosaccharide library synthesis

### Experimentation

Glycosylation reactions were broadly divided into four categories according to the reactivities of thioglycosyl donors and thioglycosyl donor/acceptors, namely, armed donor (e.g. 1-3) with armed donor/acceptor (e.g. 4, 5), disarmed donor (e.g. 6) with armed donor/acceptor, armed donor with disarmed donor/acceptor (e.g. 7, 8), and disarmed donor with disarmed donor/acceptor. After much experimentation, a general reaction condition was established for all four classes of reactions, utilizing p-tolyl thioglycosides as building blocks, TolSOTf, formed in situ by p-toluenesulfenyl chloride (TolSCl) and silver triflate (AgOTf), as the stoichiometric promoter, diethyl ether as the solvent in the presence of MS-AW300 as the dehydrating reagent. In all cases, chemoselective glycosylation of donors were observed regardless of their activities (Table 1).

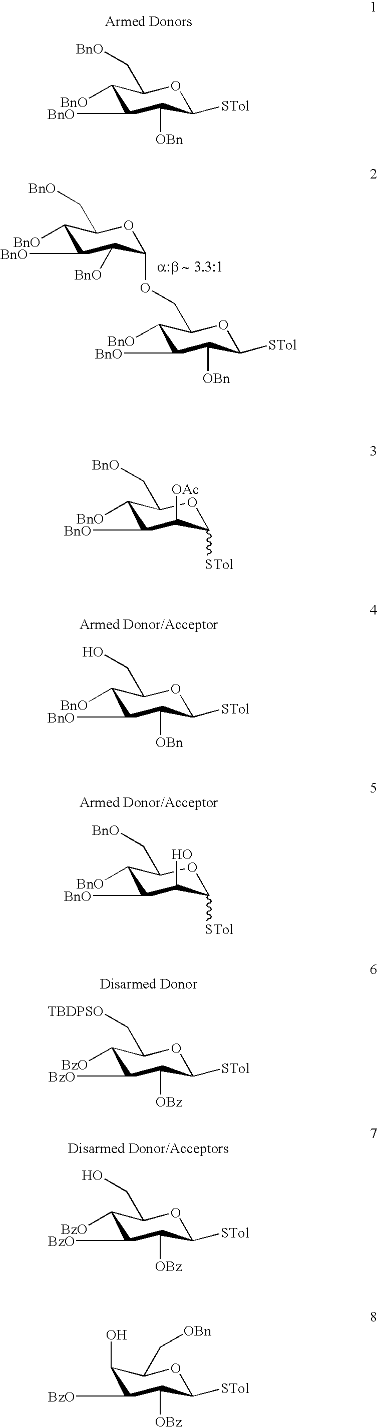

Addition of 1 eq of TolSCl to a solution of armed donor 1 and AgOTf in diethyl ether in the presence of MS-AW300 at −60° C. led to complete activation of donor 1, to which a solution of the armed donor/acceptor 4 in ether was then added. Disaccharide 2 bearing anomeric thiotolyl was obtained in 74% yield with an α:β ratio of 3.3:1 (Table 1, entry 1). Disaccharide 2 was subsequently glycosylated with donor/acceptor 4 to produce the trisaccharide 9 in 71% yield (entry 2). Armed donor/acceptor 5 bearing an axial hydroxyl group readily reacted with donors 1 and 3 to generate disaccharides 10 and 11 both in 66% yield (entries 3 and 4).

TolSOTf is a powerful promoter, capable of stoichiometrically activating disarmed donors as well. Disarmed donor 6 reacted smoothly with the more reactive armed donor/receptor 4 to give disaccharide 12 in 69% as the β anomer (entry 5). No products due to the self coupling of donor/acceptor 4 were isolated. This reversal of reactivity, i.e., less reactive donor is selectively glycosylated with the more reactive donor/acceptor, is not possible with the traditional reactivity based one-pot approach.

TABLE 1
Results of Chemoselective Glycosylations of Thioglycoside Donors

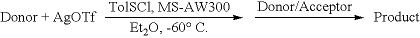

| | Donor + | | Yield [%] |
|---|---|---|---|
| Entry | Donor/Acceptor | Product | (α:β)^{a} |
| 1 | 1 + 4 | 2 | 74 (3.3:1) |
| 2 | 2 + 4 | 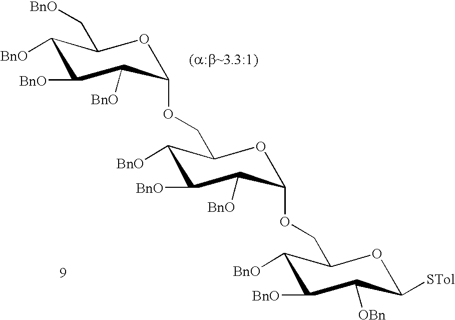 | 71 (α) |
| 3 | 1 + 5 | 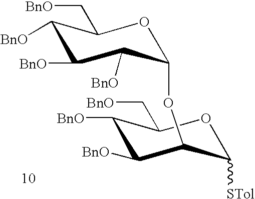 | 66 (2.3:1) |
| 4 | 3 + 5 | 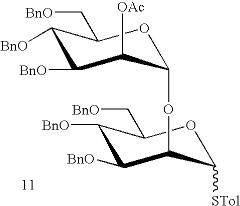 | 66 (α) |
| 5 | 6 + 4 | 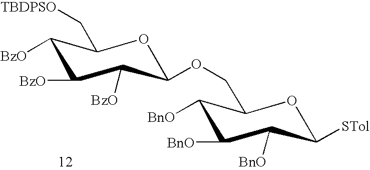 | 69 (β) |
| 6 | 1 + 7 | 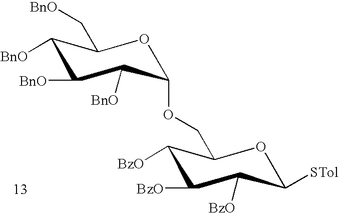 | 72 (α) |
| 7 | 6 + 7 | 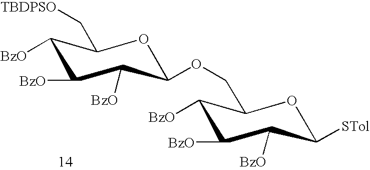 | 67 (β) |
| 8 | 1 + 8 | 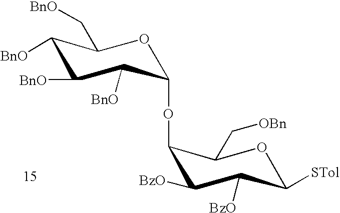 | 65 (α) |

^{a}α:β ratio refers to the anomeric distribution of the newly formed glycosidic bond.

In order to explore the scope of the current method, glycosylations with less nucleophilic disarmed donor/acceptors were examined, which are known to give low glycosylation yields due to competition of other more nucleophilic compounds in the reaction mixture. The disarmed donor/acceptor 7 was glycosylated smoothly by both pre-activated armed donor 1 and disarmed donor 6 in 72% and 67% yield respectively (entries 6 and 7). Donor 1 facilely reacted with a poorly-nucleophilic disarmed donor/acceptor 8 to give disaccharide 15 in 65% yield (entry 8).

To demonstrate the feasibility of performing multiple step glycosylations in one-pot regardless of donor reactivities, we have synthesized several oligosaccharides 9, 17, 19, 20 and 24 (Scheme 5) by sequential addition of building blocks to the same reaction vessel. It is worth noting that the yield for the one-pot synthesis of trisaccharide 9 is 66% (Scheme 5a), while overall yield through stepwise synthesis is only 53% (Table 1, entries 1,2). Thus, one-pot synthesis not only removes the need to purify the intermediate, but also leads to higher overall yield presumably due to fewer purification steps. Furthermore, the second and third building blocks utilized in this synthesis are identical for the same Glc-α-1,6-Glc linkage. For reactivity based one-pot synthesis, building blocks with different reactivities would have to be prepared to accomplish this.

The syntheses of oligosaccharides 17, 19, 20 and 24, which were achieved in 68%, 55%, 48% and 59% respective overall yields, demonstrate the assembly of oligosaccharides with several different building blocks in one-pot (Scheme 5b, 5c, 5d and 5e). The progress of our one-pot reaction can be readily monitored by TLC, allowing correction of syntheses at any unproductive coupling steps. The final oligosaccharide products from all one-pot syntheses were readily purified by silica-gel flash chromatography, which was the only purification step necessary. The trisaccharide 24 was subsequently deprotected in 70% yield to give trisaccharide 25 demonstrating that standard protective groups such as benzoate, phthalimide, and benzyl in the oligosaccharide product obtained by iterative one-pot synthesis can be efficiently removed (Scheme 5f).

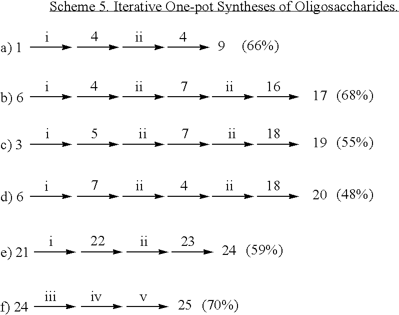

Reagents and conditions: i) AgOTf (5 eq), TolSCl (1 eq), Et₂O, MS-AW300, −60° C.; ii) TolSCl (1 eq); iii) ethylene diamine, EtOH, reflux; iv) Ac₂O, MeOH; v) H₂, Pd/C

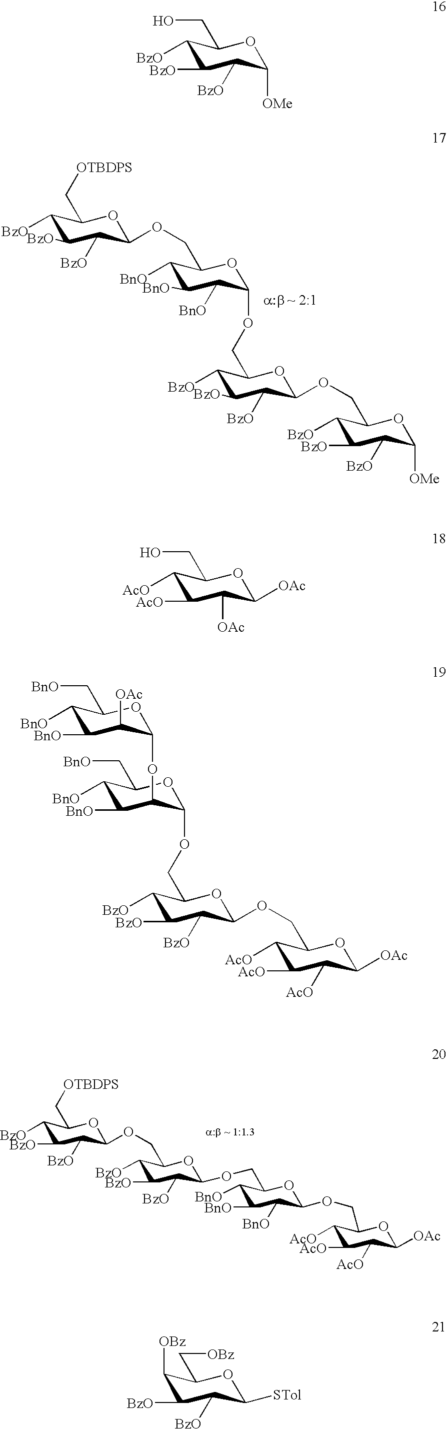

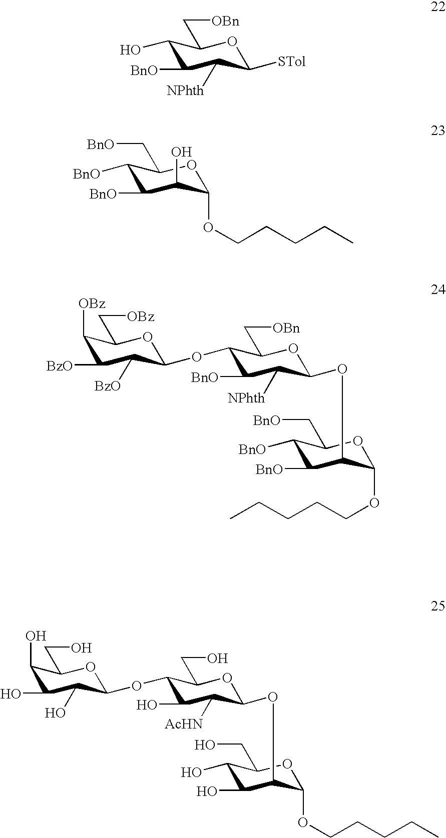

Additional work has led to the development of a fluorous catch-and-release protocol for purification of the desired oligosaccharide, an example of which is shown in Scheme 6.

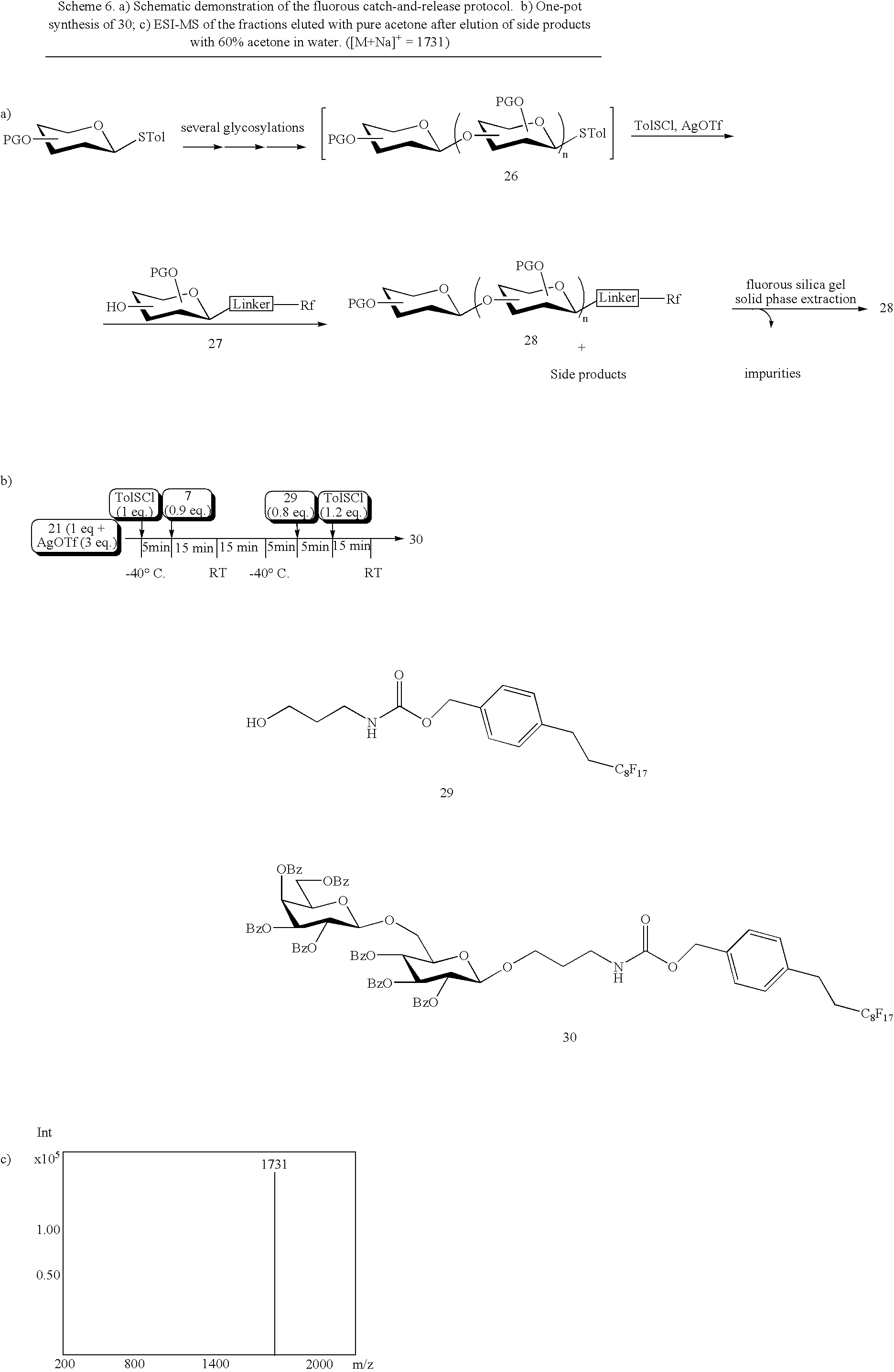

A fluorous affinity tag can be incorporated on the final acceptor (e.g. 27). Fluorous groups have high affinities towards fluorinated surfaces, which can have a role in immobilization of oligosaccharides. Upon completion of the last glycosylation reaction with the fluorous tagged final acceptor, the major product in the reaction mixture will be the desired oligosaccharide containing the fluorous tag at the reducing end (e.g. 28) (Scheme 6a). The amount of deletion sequence bearing the tag is reduced by using a temperature cycling protocol. The reaction mixture is subjected to fluorous solid phase extraction (SPE) on a fluorous silica gel cartridge. Excess reagents and side products can be readily removed by washing with a fluorophobic solvent (e.g. 60% acetone in water). Only compound containing the fluorous moiety, which is mainly the desired product (i.e., 28), will be selectively caught on the fluorous silica gel and thus be separated from all side products. Subsequent elution with a more fluorophilic solvent (e.g. pure acetone) allows facile release of the desired product. The oligosaccharide can be further purified by silica gel chromatography if necessary and the fluorous SPE can be easily automated via the use of 96 well plates. As a proof of the principle, one-pot sequential glycosylation of thiogalactoside 21, glucoside 7 and a fluorinated acceptor 29 yielded disaccharide 30, which was easily isolated using fluorous SPE (Scheme 6b, 6c). No deletion sequences were detected by ESI-MS.

In accordance with the provisions of the patent statutes, the principle and mode of operation of this invention have been explained and illustrated in its preferred embodiments. However, it must be understood that this invention may be practiced otherwise than as specifically explained and illustrated without departing from its spirit or scope.

1. A process for synthesizing an oligosaccharide, which contains three or more glycosyl units linked to one another by glycosidic linkages, starting with a first glycosyl unit at a nonreducing end, concluding with a final glycosyl unit at a reducing end, and including one or more intermediate glycosyl units sequentially arrayed between the first and final glycosyl units, the process comprising the steps of:
(a) synthesizing a protected glycosyl donor corresponding to the first glycosyl unit, one or more protected glycosyl donor/acceptors corresponding to each of the intermediate glycosyl units, and a protected glycosyl acceptor corresponding to the final glycosyl unit, the protected glycosyl donor having an activatable aglycon at the anomeric carbon, the protected glycosyl donor/acceptors each having both an activatable aglycon at the anomeric carbon and a free hydroxyl group, the glycosyl acceptor having a free hydroxyl group and a non-activatable aglycon at the anomeric carbon;
(b) activating the glycosyl donor with a promoter in the absence of a glycosyl acceptor to produce a reactive intermediate;
(c) adding one of the protected glycosyl donor/acceptors to the reactive intermediate to produce a new glycosyl donor;
(d) repeating steps (b) and (c) to add any additional protected glycosyl donor/acceptors; and
(e) adding the protected glycosyl acceptor to produce the oligosaccharide.

2. A process according to claim 1 comprising additional steps of removing all protective groups from the protected oligosaccharide.

3. A process according to claim 1 comprising an additional step of purifying the desired oligosaccharide.

4. A process according to claim 1 wherein the glycosyl units are thioglycosides, glycosyl iodides, glycosyl fluorides, or glycosyl sulfoxides.

5. A process according to claim 1 wherein the thiophilic promoter is utilized for thioglycoside building blocks.

6. A process according to claim 1 wherein the promoter is stoichiometric in activating the glycosyl donors.

7. A process according to claim 1 wherein the process is substantially unaffected by anomeric reactivities of the glycosyl donors and the one or more glycosyl donor/acceptors.

8. A process according to claim 1 which excludes any purification steps besides purification of the final oligosaccharide product.

9. A process according to claim 1 which excludes any protective group adjustments or any aglycon modifications of the products of steps (b) through (d).

10. A process according to claim 1 which is useful for synthesizing a wide variety of oligosaccharides including linear and branched oligosaccharides.

11. A process for synthesizing an oligosaccharide, which contains three or more glycosyl units linked to one another by glycosidic linkages, starting with a first glycosyl unit at a nonreducing end, concluding with a final glycosyl unit at a reducing end, and including one or more intermediate glycosyl units sequentially arrayed between the first and final glycosyl units, the process comprising the steps of:
(a) activating a protected glycosyl donor with a promoter in the absence of a glycosyl acceptor to produce a reactive intermediate, the glycosyl donor corresponding to the first glycosyl unit and having an activatable aglycon at the anomeric carbon;
(b) adding a protected glycosyl donor/acceptor to the reactive intermediate to produce a new glycosyl donor, the glycosyl donor/acceptor corresponding to one of the one or more intermediate glycosyl units and having both an activatable aglycon at the anomeric carbon and a free hydroxyl group;
(c) repeating steps (a) and (b) to add any additional protected glycosyl donor/acceptors corresponding to the one or more intermediate glycosyl units; and
(d) adding a protected glycosyl acceptor to produce the oligosaccharide, the glycosyl acceptor corresponding to the final glycosyl unit and having a free hydroxyl group and a non-activatable aglycon at the anomeric carbon.

12. A process for synthesizing an oligosaccharide and facilitating the purification of the oligosaccharide, the oligosaccharide containing three or more glycosyl units linked to one another by glycosidic linkages, starting with a first glycosyl unit at a nonreducing end, concluding with a final glycosyl unit at a reducing end, and including one or more intermediate glycosyl units sequentially arrayed between the first and final glycosyl units, the process comprising the steps of:
(a) synthesizing a protected glycosyl donor corresponding to the first glycosyl unit, one or more protected glycosyl donor/acceptors corresponding to each of the intermediate glycosyl units, and a protected glycosyl acceptor corresponding to the final glycosyl unit, the synthesizing of the glycosyl acceptor including a step of incorporating an affinity tag;
(b) activating the glycosyl donor with a promoter in the absence of a glycosyl acceptor to produce a reactive intermediate;
(c) adding one of the protected glycosyl donor/acceptors to the reactive intermediate to produce a new glycosyl donor;
(d) repeating steps (b) and (c) to add any additional protected glycosyl donor/acceptors; and
(e) adding the protected glycosyl acceptor to produce the oligosaccharide, the oligosaccharide including the affinity tag at the reducing end, the affinity tag facilitating purification of the oligosaccharide.

13. A process according to claim 12 wherein steps (b) through (e) are carried out in solution, and comprising an additional step of adding the polymer to the solution such that the oligosaccharide attaches to the polymer.

14. A process according to claim 13 wherein the polymer is added in the form of polymer beads.

15. A process according to claim 13 comprising the additional steps of isolating the polymer from the solution, and removal of the oligosaccharide from the polymer.

16. A process according to claim 12 wherein the affinity tag is a fluorous affinity tag.

17. A process for synthesizing an oligosaccharide library attached to polymer comprising the steps of:
(a) synthesizing a set of protected glycosyl donors, protected glycosyl donor/acceptors and protected glycosyl acceptors from different monosaccharides, the glycosyl donor/acceptors having at least one of 2-OH, 3-OH, 4-OH and 6-OH unprotected and glycosyl acceptors having an affinity tag and at least one of 2-OH, 3-OH, 4-OH and 6-OH unprotected;
(b) activating one of the glycosyl donors with a promoter in the absence of a glycosyl acceptor to produce a reactive intermediate;
(c) adding one of the protected glycosyl donor/acceptors to the reactive intermediate to produce a new glycosyl donor;
(d) repeating steps (b) and (c) to add any additional protected glycosyl donor/acceptors desired in a first oligosaccharide;
(e) adding one of the protected glycosyl acceptors to produce the first oligosaccharide;
(f) adding a polymer to attach the affinity tag containing oligosaccharide product; and
(g) repeating steps (b) through (f) using other combinations of the glycosyl donors, glycosyl donor/acceptors and glycosyl acceptors to produce other members of the oligosaccharide library on the polymer.

18. A process for synthesizing an oligosaccharide library comprising the steps of:
(a) synthesizing a set of protected glycosyl donors, protected glycosyl donor/acceptors and protected glycosyl acceptors from different monosaccharides, the glycosyl donor/acceptors and glycosyl acceptors having at least one of 2-OH, 3-OH, 4-OH and 6-OH unprotected;
(b) activating one of the glycosyl donors with a promoter in the absence of a glycosyl acceptor to produce a reactive intermediate;
(c) adding one of the protected glycosyl donor/acceptors to the reactive intermediate to produce a new glycosyl donor;
(d) repeating steps (b) and (c) to add any additional protected glycosyl donor/acceptors desired in a first oligosaccharide;
(e) adding one of the protected glycosyl acceptors to produce the first oligosaccharide; and
(f) repeating steps (b) through (e) using other combinations of the glycosyl donors, glycosyl donor/acceptors and glycosyl acceptors to produce other members of the oligosaccharide library.
